(19) United States
(12) Patent Application Publication
(10) Pub. No.: US 2003/0172472 A1
(43) Pub. Date: Sep. 18, 2003

(54) Oxidaton dyeing composition for keratinous fibres comprising an amphoteric polymer with fatty chain

(76) Inventor: Florence Laurent, Asnieres (FR)

Correspondence Address: FINNEGAN, HENDERSON, FARABOW, GARRETT & DUNNER, LLP, 1300 I STREET, NW, WASHINGTON, DC, 20005, US

(21) Appl. No.: 10/312,628

(22) PCT Filed: Jun. 29, 2001
(86) PCT No.: PCT/FR01/02095

(30) Foreign Application Priority Data
Jun. 30, 2000 (FR) 00/08503

(51) Int. Cl. A61K 7/13

(52) U.S. Cl. 8/405; 8/406; 8/408; 8/415; 8/421

(57) ABSTRACT

The invention relates to a composition for the oxidation dyeing of keratin fibres, particularly human keratin fibres such as hair, comprising, in an appropriate dyeing medium, at least one oxidation dyestuff precursor and/or coupler selected from paraphenylenediamines, double bases, paraminophenols, metaphenylenediamines, metaminophenols, metadiphenols, bases and heterocyclic couplers, and the addition salts of these compounds with an acid, and at least one amphoteric polymer with at least one fatty chain having from 8 to 30 carbon atoms.

The invention further relates to the dyeing processes and devices in which said oxidation dyeing composition is used.

The present invention relates to a composition for the oxidation dyeing of keratin fibres, particularly human keratin fibres such as hair, comprising at least one oxidation dyestuff precursor and/or specific coupler and at least one amphoteric polymer with at least one fatty chain.

It is known to dye keratin fibres, particularly human hair, with dyeing compositions containing oxidation dyestuffs and especially oxidation dyestuff precursors—generally called “oxidation bases”—particularly ortho- or paraphenylenediamines, ortho- or paraminophenols, and heterocyclic bases.

Oxidation dyestuff precursors are compounds with little or no initial colour which develop their tinctorial strength inside the hair in the presence of oxidizing agents to form coloured compounds. The formation of these coloured compounds results either from an oxidative condensation of the “oxidation bases” with themselves, or from an oxidative condensation of the “oxidation bases” with other, colouration-modifying oxidation dyestuffs, or “couplers”, which are generally present in the dyeing compositions used in oxidation dyeing and are represented more particularly by metaphenylenediamines, metaminophenols and metadiphenols, and certain heterocyclic compounds.

The variety of molecules used, which consist on the one hand of “oxidation bases” and on the other hand of “couplers”, affords a very rich palette of shades.

The so-called “permanent” colouration obtained by means of these oxidation dyestuffs also has to satisfy a number of requirements. Thus it has to afford the desired degrees of intensity and have a good resistance to external agents (light, weather, washing, perming, perspiration, rubbing).

The dyestuffs must also be capable of covering white hair and, finally, must have the lowest possible selectivity, i.e. they must afford the smallest possible differences in colouration along one and the same keratin fibre, which can actually be differently sensitized (i.e. damaged) between its tip and its root.

Apart from traditional thickeners such as crosslinked polyacrylic acid, hydroxyethyl celluloses, waxes and mixtures of non-ionic surfactants of appropriately chosen HLB (Hydrophilic Lipophilic Balance), associative polymers of the anionic, non-ionic or cationic type have also been used for localizing the oxidation dye on application to the hair so that it does not run onto the face or beyond the zones which it is proposed to dye.

However, the traditional thickening systems, namely waxes and said mixtures of surfactants mentioned above, do not make it possible to obtain strong and chromatic shades of low selectivity and good resistance while at the same time ensuring that the treated hair is in good cosmetic condition. Furthermore, the ready-to-use dyeing compositions containing the oxidation dyestuff(s) and said thickening systems of the prior art do not allow a sufficiently precise application without the composition running and without its viscosity falling over time.

The associative polymers of the anionic, non-ionic or cationic type represent an important advance in the search for a solution to this problem.

The Applicant has now discovered, totally unexpectedly and surprisingly, novel oxidation dyeing compositions (after mixing with the oxidizing agent) which do not run and therefore remain better localized at the point of application, and which afford strong and chromatic shades of low selectivity and good resistance while at the same time ensuring that the treated hair is in good cosmetic condition, said dyeing compositions comprising an effective amount of an amphoteric polymer with at least one fatty chain which is introduced either (i) into the composition containing the oxidation dyestuff precursor(s) and/or specific couplers, or (ii) into the oxidizing composition, or (iii) into both compositions at once.

This discovery forms the basis of the present invention.

The present invention thus relates to a novel composition for the oxidation dyeing of keratin fibres, particularly human keratin fibres such as hair, comprising, in an appropriate dyeing medium, at least one oxidation dyestuff precursor and/or coupler selected from paraphenylenediamines, double bases, paraminophenols, metaphenylenediamines, metaminophenols, metadiphenols, bases and heterocyclic couplers, and the addition salts of these compounds with an acid, and at least one amphoteric polymer with at least one fatty chain having from 8 to 30 carbon atoms.

The present invention further relates to a ready-to-use composition for the dyeing of keratin fibres which contains at least one oxidation dyestuff precursor and/or coupler as defined above and at least one amphoteric polymer with at least one fatty chain, and also an oxidizing agent.

In terms of the present invention, “ready-to-use composition” is understood as meaning any composition intended for direct application to keratin fibres, and “fatty chain” is understood as meaning a chain having from 8 to 30 carbon atoms.

The invention further relates to a process for dyeing keratin fibres, particularly human keratin fibres such as hair, which consists in applying to the fibres at least one dyeing composition containing, in an appropriate dyeing medium, at least one oxidation dyestuff precursor and/or coupler as defined above, the colour being developed at alkaline, neutral or acidic pH with the aid of an oxidizing composition containing at least one oxidizing agent, which is mixed with the dyeing composition at the time of use or is applied sequentially without intermediate rinsing, at least one amphoteric polymer with at least one fatty chain being present in the dyeing composition, in the oxidizing composition or in each of said compositions.

The invention further relates to dyeing devices or kits comprising several compartments.

A 2-compartment device according to the invention consists of one compartment comprising a dyeing composition containing, in an appropriate dyeing medium, at least one oxidation dyestuff precursor and/or coupler as defined above, and another compartment comprising an oxidizing composition containing, in an appropriate dyeing medium, an oxidizing agent, the amphoteric polymer with at least one fatty chain being present in the dyeing composition, in the oxidizing composition or in each of these compositions.

However, further characteristics, features, objects and advantages of the invention will become even more clearly apparent from the description and examples which follow.

Amphoteric Polymers With at Least One Fatty Chain Having from 8 to 30 Carbon Atoms

“Amphoteric polymers” are generally understood as meaning polymers containing units K and M randomly distributed along the polymer chain, where K denotes a unit derived from a monomer containing at least one basic nitrogen atom and M denotes a unit derived from an acidic monomer containing one or more carboxylic or sulphonic acid groups, or alternatively K and M can denote groups derived from zwitterionic monomers of carboxybetaines or sulphobetaines;

K and M can also denote a cationic polymer chain containing primary, secondary, tertiary or quaternary amine groups, in which at least one of the amine groups carries a carboxylic or sulphonic acid group joined via a hydrocarbon radical, or alternatively K and M form part of a chain of a polymer with an α,β-dicarboxyethylene unit in which one of the carboxyl groups has been reacted with a polyamine containing one or more primary or secondary amine groups.

The amphoteric polymers used according to the invention also contain at least one fatty chain having from 8 to 30 carbon atoms and can be selected for example from polymers derived from polyaspartic acid that contain at least one fatty chain having from 8 to 30 carbon atoms, such as:

those described and prepared in patent application EP-0 767 191, the content of which forms an integral part of the present invention. Such polymers are prepared in known manner by the reaction of polysuccinamide (PSI) with amines containing a fatty chain (C₈-C₂₄), in a solvent medium, in the presence or absence of a basic catalyst, for example aliphatic tertiary amines, followed by amphoterization of the resulting product by reaction with a halogenated organic acid.

The following may be mentioned in particular among the amines with a C₈-C₂₄ fatty chain that are reacted with PSI: octylamine, nonylamine, decylamine, dodecylamine, tetradecylamine, hexadecylamine, octadecylamine, octadecenylamine, eicosyldecylamine, octynylamine, decenylamine, dodecenylamine, tetradecenylamine, hexadecenylamine, octadecenylamine and eicosenylamine.

Examples of such polymers are prepared by the reaction of PSI with n-laurylamine or n-stearylamine in the presence of N,N-dimethyl-1,3-propanediamine as basic catalyst, followed by amphoterization of the resulting product by reaction with potassium monochloroacetate. The preparation of these polymers is described in greater detail on pages 13 to 20 (lines 1-4) and in Examples 1 to 5 on pages 28 to 34 (lines 1-4) of said patent application EP-0 767 191.

those described and prepared in patent application EP-0 884 344, the content of which forms an integral part of the present invention. Such polymers are prepared by the reaction of gaseous ammonia with a C₈-C₂₄ alkyl or alkenyl monomaleate in a solvent medium under reduced pressure and at a temperature of 120-140° C. for 4 to 6 hours.

The C₈-C₂₄ alkyl or alkenyl radicals can be chosen in particular from the following linear or branched radicals: decyl, dodecyl, tetradecyl, hexadecyl, octadecyl and oleyl.

Examples of such polymers include polymers with aspartic acid units and decyl aspartate units, polymers with aspartic acid units and dodecyl aspartate units, polymers with aspartic acid units and cetyl aspartate units, polymers with aspartic acid units and stearyl aspartate units and polymers with aspartic acid units and n-decylaspartamide units, described in Examples 1 to 6 of said patent application.

those described and prepared in patent application EP-0 959 094, the content of which forms an integral part of the present invention. Such polymers are prepared by the reaction of gaseous ammonia, in a solvent medium, with a polyalkoxylated maleic acid monoamide rendered hydrophobic with a linear or branched C₈-C₃₀ alkyl or alkenyl chain, optionally mixed with a maleic acid monoester.

An example of a polymer prepared in this way is described in Example 2 on page 11 of said patent application.

those described and prepared in patent application EP-0 959 090, the content of which forms an integral part of the present invention. Such high-molecular-weight polymers which have been rendered hydrophobic are obtained from maleic acid derivatives and gaseous ammonia and from difunctional or polyfunctional alcohols or amines.

Examples of copolymers with aspartic acid and cetyl aspartate units or with aspartic acid and cetyl aspartate units are given respectively in Examples 3 and 5 of said patent application.

or those described and prepared in patent application EP-0 959 091, the content of which forms an integral part of the present invention. Such polymers which have been rendered hydrophobic are prepared from a maleic acid monoester or monoamide and gaseous ammonia.

Examples of such copolymers are given in Examples 1, 2, 3 and 5 of said patent application.

Preferably, according to the present invention, the amphoteric polymers with at least one fatty chain having from 8 to 30 carbon atoms are selected from those containing at least one non-cyclic cationic unit. Those prepared from or comprising 1 to 20 mol % of monomer with a fatty chain, preferably 1.5 to 15 mol % and particularly preferably 1.5 to 6 mol %, based on the total number of moles of monomers, are more particularly preferred.

Said amphoteric polymers with a fatty chain which are preferred according to the invention comprise or are prepared by copolymerizing:

1) at least one monomer of formula (Ia) or (Ib):

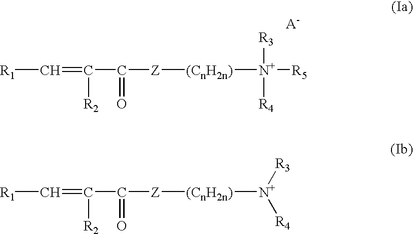

in which:

R₁ and R₂, which are identical or different, are a hydrogen atom or a methyl radical;

R₃, R₄ and R₅, which are identical or different, are a linear or branched alkyl radical having from 1 to 30 carbon atoms;

Z is an NH group or an oxygen atom;

n is an integer from 2 to 5; and

A⁻ is an anion derived from an organic or mineral acid, such as a methosulphate anion or a halide such as chloride or bromide;

2) at least one monomer of formula (II):

R₆—CH═CR₇—COOH (II)

in which:

R₆ and R₇, which are identical or different, are a hydrogen atom or a methyl radical; and

3) at least one monomer of formula (III):

R₆—CH═CR₇—COXR₈ (III)

in which:

R₆ and R₇, which are identical or different, are a hydrogen atom or a methyl radical;

X denotes an oxygen or nitrogen atom; and

R₈ denotes a linear or branched alkyl radical having from 1 to 30 carbon atoms,

at least one of the monomers of formula (Ia), (Ib) or (III) containing at least one fatty chain.

The monomers of formulae (Ia) and (Ib) of the present invention are preferably selected from the group comprising:

dimethylaminoethyl methacrylate, dimethylaminoethyl acrylate,

diethylaminoethyl methacrylate, diethylaminoethyl acrylate,

dimethylaminopropyl methacrylate, dimethylaminopropyl acrylate, and

dimethylaminopropylmethacrylamide, dimethylaminopropylacrylamide, these

monomers optionally being quaternized, for example with a C₁-C₄ alkyl halide or a C₁-C₄ dialkyl sulphate.

More particularly, the monomer of formula (Ia) is selected from acrylamidopropyltrimethylammonium chloride and methacrylamidopropyltrimethylammonium chloride.

The monomers of formula (II) of the present invention are preferably selected from the group comprising acrylic acid, methacrylic acid, crotonic acid and 2-methylcrotonic acid. More particularly, the monomer of formula (II) is acrylic acid.

The monomers of formula (III) of the present invention are preferably selected from the group comprising C₁₂-C₂₂ and more particularly C₁₆-C₁₈ alkyl acrylates or methacrylates.

The monomers constituting the amphoteric polymers with a fatty chain of the invention are preferably already neutralized and/or quaternized.

The ratio of the number of cationic charges to anionic charges is preferably equal to about 1.

The amphoteric polymers with a fatty chain according to the invention preferably comprise from 1 to 10 mol %, particularly preferably from 1.5 to 6 mol %, of monomer with a fatty chain (monomer of formula (Ta), (Ib) or (III)).

The weight-average molecular weights of the amphoteric polymers with a fatty chain according to the invention can vary from 500 to 50,000,000 and are preferably between 10,000 and 5,000,000.

The amphoteric polymers with a fatty chain according to the invention can also contain other monomers such as non-ionic monomers and particularly such as C₁-C₄ alkyl acrylates or methacrylates.

Amphoteric polymers with a fatty chain according to the invention are described and prepared for example in patent application WO 98/44012.

Preferred amphoteric polymers with a fatty chain according to the invention are acrylic acid/(meth)acrylamidopropyltrimethylammonium chloride/stearyl methacrylate terpolymers.

The amphoteric polymer(s) with a fatty chain according to the invention is (are) preferably used in an amount of between about 0.05 and 10% by weight, based on the total weight of the composition. This amount is more preferably between about 0.1 and 5% by weight, based on the total weight of the composition.

Oxidation Dyestuff Precursors

(I) The paraphenylenediamines are selected in particular from those of formula (I) below and their acid addition salts:

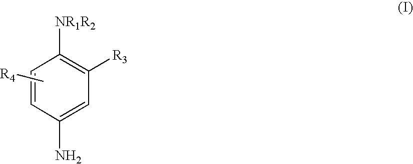

in which:

R₁ is a hydrogen atom, a C₁-C₄ alkyl radical, a C₁-C₄ monohydroxyalkyl radical, a C₂-C₄ polyhydroxyalkyl radical, an alkoxy(C₁-C₄)alkyl(C₁-C₄) radical or a C₁-C₄ alkyl radical substituted by a nitrogen, phenyl or 4′-aminophenyl group;

R₂ is a hydrogen atom, a C₁-C₄ alkyl radical, a C₁-C₄ monohydroxyalkyl radical, a C₂-C₄ polyhydroxyalkyl radical, an alkoxy(C₁-C₄)alkyl(C₁-C₄) radical or a C₁-C₄ alkyl radical substituted by a nitrogen group;

R₃ is a hydrogen atom, a halogen atom such as a chlorine atom, or a C₁-C₄ alkyl, sulpho, carboxyl, C₁-C₄ monohydroxyalkyl, C₁-C₄ hydroxyalkoxy, C₁-C₄ acetylaminoalkoxy, C₁-C₄ mesylaminoalkoxy or C₁-C₄ carbamoylaminoalkoxy radical; and

R₄ is a hydrogen or halogen atom or a C₁-C₄ alkyl radical,

it also being possible for R₁ and R₂ to form, with the nitrogen atom which carries them, a 5- or 6-membered nitrogen heterocycle optionally substituted by one or more alkyl, hydroxyl or ureido groups.

Amino, monoalkyl (C₁-C₄) amino, dialkyl(C₁-C₄)amino, trialkyl (C₁-C₄) amino, monohydroxyalkyl(C₁-C₄) amino, imidazolinium and ammonium radicals may be mentioned in particular among the nitrogen groups of formula (I) above.

The following may be mentioned more particularly among the paraphenylenediamines of formula (I) above: paraphenylenediamine, paratoluylenediamine, 2-chloroparaphenylenediamine, 2,3-dimethylparaphenylenediamine, 2,6-dimethylparaphenylenediamine, 2,6-diethylparaphenylenediamine, 2,5-dimethylparaphenylenediamine, N,N-dimethylparaphenylenediamine, N,N-diethylparaphenylenediamine, N,N-dipropylparaphenylenediamine, 4-amino-N,N-diethyl-3-methylaniline, N,N-bis(β-hydroxyethyl)paraphenylenediamine, 4-N,N-bis(β-hydroxyethyl)-amino-2-methylaniline, 4-N,N-bis(β-hydroxyethyl)amino-2-chloroaniline, 2-β-hydroxyethylparaphenylenediamine,

2-fluoroparaphenylenediamine, 2-isopropylparaphenylenediamine, N—(β-hydroxypropyl)paraphenylenediamine, 2-hydroxymethylparaphenylenediamine, N,N-dimethyl-3-methylparaphenylenediamine, N,N-(ethyl,β-hydroxyethyl)-paraphenylenediamine, N-(β,γ-dihydroxypropyl)paraphenylenediamine, N-(4′-aminophenyl) paraphenylenediamine, N-phenylparaphenylenediamine, 2-β-hydroxyethyloxyparaphenylenediamine, 2-β-acetylaminoethyloxyparaphenylenediamine, N-(β-methoxyethyl)paraphenylenediamine, 2-methyl-1-N-β-hydroxyethylparaphenylenediamine and their acid addition salts.

The following are very particularly preferred among the paraphenylenediamines of formula (I) above: paraphenylenediamine, paratoluylenediamine, 2-isopropylparaphenylenediamine, 2-β-hydroxyethylparaphenylenediamine, 2-β-hydroxyethoxyparaphenylenediamine, 2,6-dimethylparaphenylenediamine, 2,6-diethylparaphenylenediamine, 2,3-dimethylparaphenylenediamine, N,N-bis(β-hydroxyethyl)paraphenylenediamine, 2-chloroparaphenylenediamine and their acid addition salts.

(II) According to the invention, “double bases” are understood as meaning compounds containing at least two aromatic rings carrying amino and/or hydroxyl groups.

The compounds of formula (II) below and their acid addition salts may be mentioned in particular among the double bases which can be used as oxidation dyestuff precursors in the dyeing compositions according to the invention:

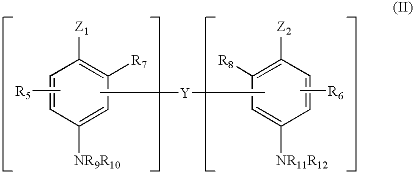

in which:

Z₁ and Z₂, which are identical or different, are a hydroxyl or —NH₂ radical which can be substituted by a C₁-C₄ alkyl radical or by a linking arm Y;

the linking arm Y is a linear or branched alkylene chain containing from 1 to 14 carbon atoms which can be interrupted or terminated by one or more nitrogen groups and/or by one or more heteroatoms such as oxygen, sulphur or nitrogen atoms, and optionally substituted by one or more hydroxyl or C₁-C₆ alkoxy radicals;

R₅ and R₆ are a hydrogen or halogen atom, a C₁-C₄ alkyl, C₁-C₄ monohydroxyalkyl, C₂-C₄ polyhydroxyalkyl or C₁-C₄ aminoalkyl radical or a linking arm Y; and

R₇, R₈, R₉, R₁₀, R₁₁ and R₁₂, which are identical or different, are a hydrogen atom, a linking arm Y or a C₁-C₄ alkyl radical,

it being understood that the compounds of formula (II) contain only one linking arm Y per molecule.

Amino, monoalkyl (C₁-C₄) amino, dialkyl(C₁-C₄) amino, trialkyl (C₁-C₄)amino, monohydroxyalkyl(C₁-C₄) amino, imidazolinium and ammonium radicals may be mentioned in particular among the nitrogen groups of formula (II) above.

The following may be mentioned more particularly among the double bases of formula (II) above: N,N′-bis (phydroxyethyl)-N,N′-bis (4′-aminophenyl)-1,3-diaminopropanol, N,N′-bis (β-hydroxyethyl) —N,N′-bis (4′-aminophenyl)ethylenediamine, N,N′-bis (4-aminophenyl)tetramethylenediamine, N,N′-bis (β-hydroxyethyl) —N,N′-bis (4-aminophenyl) tetramethylenediamine, N,N′-bis (4-methylaminophenyl)tetramethylenediamine, N,N′-bis (ethyl) —N,N′-bis (4′-amino, 3′-methylphenyl)ethylenediamine, 1,8-bis (2,5-diaminophenoxy)-3,5-dioxaoctane and their acid addition salts.

N,N′-bis (β-hydroxyethyl) —N,N′-bis (4′-aminophenyl)-1,3-diaminopropanol, 1,8-bis(2,5-diaminophenoxy)-3,5-dioxaoctane or one of their acid addition salts are particularly preferred among these double bases of formula (II).

(III) The paraminophenols are selected in particular from those of formula (III) below and their acid addition salts:

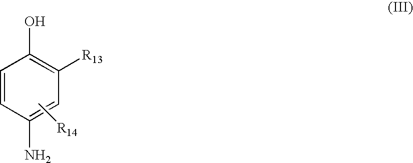

in which:

R₁₃ is a hydrogen atom, a halogen atom such as fluorine, or a C₁-C₄ alkyl, C₁-C₄ monohydroxyalkyl, alkoxy(C₁-C₄)— alkyl (C₁-C₄), C₁-C₄ aminoalkyl or hydroxyalkyl(C₁-C₄)— aminoalkyl(C₁-C₄) radical; and

R₁₄ is a hydrogen atom, a halogen atom such as fluorine, or a C₁-C₄ alkyl, C₁-C₄ monohydroxyalkyl, C₂-C₄ polyhydroxyalkyl, C₁-C₄ aminoalkyl, C₁-C₄ cyanoalkyl or alkoxy(C₁-C₄)alkyl(C₁-C₄) radical.

The following may be mentioned more particularly among the paraminophenols of formula (III) above: paraminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and their acid addition salts.

(IV) The following may be mentioned more particularly among the heterocyclic bases which can be used as oxidation dyestuff precursors in the dyeing compositions according to the invention: pyridine derivatives, pyrimidine and pyrazolopyrimidine derivatives, pyrazole derivatives and their acid addition salts.

Compounds which may be mentioned more particularly among the pyridine derivatives are those described e.g. in patents GB 1 026 978 and GB 1 153 196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine and their acid addition salts.

Compounds which may be mentioned more particularly among the pyrimidine derivatives are those described e.g. in German patent DE 2 359 399, Japanese patents JP 88-169 571 and JP 91-10659 or patent application WO 96/15765, such as 2,4,5,6-tetraminopyrimidine, 4-hydroxy-?,5,6-triaminopyrimidine, 2-hydroxy-4,5,6-triaminopyrimidine, 2,4-dihydroxy-5,6-diaminopyrimidine and 2,5,6-triaminopyrimidine, and compounds which may be mentioned more particularly among the pyrazolopyrimidine derivatives are those mentioned in patent application FR-A-2 750 048, such as pyrazolo[1,5-a]-pyrimidine-3,7-diamine, 2,5-dimethylpyrazolo[1,5-a]-pyrimidine-3,7-diamine, pyrazolo[1,5-a]pyrimidine-3,5-diamine, 2,7-dimethylpyrazolo[1,5-a]pyrimidine-3,5-diamine, 3-aminopyrazolo[1,5-a]pyrimidin-7-ol, 3-aminopyrazolo[1,5-a]pyrimidin-5-ol, 2-(3-aminopyrazolo-[1,5-a]pyrimidin-7-ylamino)ethanol, 2-(7-aminopyrazolo-[1,5-a]pyrimidin-3-ylamino)ethanol, 2-[(3-amino-pyrazolo[1,5-a]pyrimidin-7-yl)(2-hydroxyethyl)amino]-ethanol, 2-[(7-aminopyrazolo[1,5-a]pyrimidin-3-yl)(2-hydroxyethyl)amino]ethanol, 5,6-dimethylpyrazolo-[1,5-a]pyrimidine-3,7-diamine, 2,6-dimethylpyrazolo-[1,5-a]pyrimidine-3,7-diamine, 2,5,N7,N7-tetramethyl-pyrazolo[1,5-a]pyrimidine-3,7-diamine, 3-amino-5-methyl-7-imidazolylpropylaminopyrazolo[1,5-a]pyrimidine and their addition salts, and their tautomeric forms if there is a tautomeric equilibrium, and their acid addition salts.

Compounds which may be mentioned more particularly among the pyrazole derivatives are those described in patents DE 3 843 892 and DE 4 133 957 and patent applications WO 94/08969, WO 94/08970, FR-A-2 733 749 and DE 195 43 988, such as 4,5-diamino-1-methylpyrazole, 3,4-diaminopyrazole, 4,5-diamino-1-(4′-chlorobenzyl)pyrazole, 4,5-diamino-1,3-dimethyl-pyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole,

4,5-diamino-1-(β-hydroxyethyl)-3-methylpyrazole, 4,5-diamino-1-(β-hydroxyethyl)pyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl) amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole and their acid addition salts.

According to the present invention, the oxidation dyestuff precursors represent preferably from about 0.0005 to 12% by weight, particularly preferably from about 0.005 to 8% by weight, based on the total weight of the composition.

Couplers

The couplers which can be used in the dyeing composition according to the invention are selected from metaphenylenediamines, metaminophenols, metadiphenols and heterocyclic couplers.

The following may be mentioned among the heterocyclic couplers: indole derivatives, indoline derivatives, sesamol and its derivatives, pyridine derivatives, pyrazolotriazole derivatives, pyrazolones, indazoles, benzimidazoles, benzothiazoles, benzoxazoles, 1,3-benzodioxoles, quinolines and their acid addition salts.

These couplers are more particularly selected from 2,4-diamino-1-(β-hydroxyethyloxy)benzene, 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2-amino-4-(β-hydroxyethylamino)-1-methoxybenzene, 1,3-diaminobenzene, 1,3-bis(2,4-diaminophenoxy)propane, sesamol, 1-amino-2-methoxy-4,5-methylenedioxybenzene, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one, 1-phenyl-3-methylpyrazol-5-one, 2-amino-3-hydroxypyridine, 3,6-dimethylpyrazolo[3,2-c]-1,2,4-triazole, 2,6-dimethylpyrazolo[1,5-b]-1,2,4-triazole and their acid addition salts.

In general, the coupler(s) represent(s) preferably from about 0.0001 to 15% by weight, particularly preferably from about 0.001 to 10%, based on the total weight of the ready-to-use dyeing composition.

The acid addition salts of these oxidation dyestuff precursors and/or couplers are selected especially from hydrochlorides, hydrobromides, sulphates, tartrates, lactates and acetates.

In one preferred embodiment, the ready-to-use dyeing composition according to the invention can also contain one or more direct dyestuffs, especially for modifying the shades by enriching them with highlights. In such cases, these direct dyestuffs can be selected in particular from neutral, cationic or anionic nitro, azo or anthraquinone dyestuffs in a proportion by weight of about 0.001 to 20%, preferably of 0.01 to 10%, based on the total weight of the composition.

The appropriate dyeing medium for the composition is preferably an aqueous medium consisting of water that can advantageously contain cosmetically acceptable organic solvents, including, more particularly, alcohols such as ethyl alcohol, isopropyl alcohol, benzyl alcohol and phenylethyl alcohol, or glycols or glycol ethers such as ethylene glycol monomethyl, monoethyl and monobutyl ethers, propylene glycol or its ethers, for example propylene glycol monomethyl ether, butylene glycol, dipropylene glycol, and diethylene glycol alkyl ethers, for example diethylene glycol monoethyl ether or monobutyl ether, in concentrations of between about 0.5 and 20% by weight, preferably of between about 2 and 10% by weight, based on the total weight of the composition.

The dyeing composition and/or the oxidizing composition can also contain more particularly at least one surfactant, preferably a non-ionic surfactant, in a proportion of at least 0.01% by weight.

These surfactants are selected from:

Non-Ionic Surfactants:

Non-ionic surfactants are again compounds that are well known per se (cf. especially “Handbook of Surfactants” by M. R. PORTER, published by Blackie & Son (Glasgow and London), 1991, pp 116-178) and their nature is not critical within the framework of the present invention. Thus, without implying a limitation, they can be selected in particular from alcohols, alpha-diols, alkylphenols or polyethoxylated or polypropoxylated fatty acids with a fatty chain having e.g. 8 to 18 carbon atoms, the number of ethylene oxide or propylene oxide groups ranging especially from 2 to 50. The following may also be mentioned: ethylene oxide/propylene oxide copolymers; condensates of ethylene oxide and propylene oxide with fatty alcohols; polyethoxylated fatty amides preferably having from 2 to 30 mol of ethylene oxide; polyglycerolated fatty amides containing an average of 1 to 5, particularly 1.5 to 4, glycerol groups; polyethoxylated fatty amines preferably having 2 to 30 mol of ethylene oxide; ethoxylated sorbitan fatty acid esters having from 2 to 30 mol of ethylene oxide; sucrose fatty acid esters; polyethylene glycol fatty acid esters; alkylpolyglycosides; N-alkylglucamine derivatives; and amine oxides such as alkyl(C₁₀-C₁₄)amine oxides or N-acylaminopropylmorpholine oxides. It is pointed out that alkylpolyglycosides are particularly suitable non-ionic surfactants within the framework of the present invention.

Anionic Surfactants:

The following may be mentioned in particular, without implying a limitation, as examples of anionic surfactants which can be used, by themselves or in mixtures, within the framework of the present invention: salts (particularly the alkali metal salts, especially sodium salts, ammonium salts, amine salts, amino alcohol salts or magnesium salts) of the following compounds: alkylsulphates, alkylethersulphates, alkylamidoethersulphates, alkylarylpolyethersulphates, monoglyceridesulphates; alkylsulphonates, alkylphosphates, alkylamidesulphonates, alkylarylsulphonates, α-olefinsulphonates, paraffinsulphonates; alkyl (C₆-C₂₄) sulphosuccinates, alkyl (C₆-C₂₄) ethersulphosuccinates, alkyl-(C₆-C₂₄) amidesulphosuccinates; alkyl (C₆-C₂₄) sulphoacetates; acyl (C₆-C₂₄) sarcosinates; and acyl (C₆-C₂₄)-glutamates. It is also possible to use alkyl(C₆-C₂₄)— polyglycoside carboxylic acid esters such as alkylglucoside citrates, alkylpolyglycoside tartrates and alkylpolyglycoside sulphosuccinates; alkylsulphosuccinamates; acylisethionates; and N-acyltaurates, the alkyl or acyl radical of all these various compounds preferably containing from 12 to 20 carbon atoms and the aryl radical preferably denoting a phenyl or benzyl group. The following may also be mentioned among other anionic surfactants which can be used: fatty acid salts such as salts of oleic, ricinoleic, palmitic and stearic acids and the acids from copra oil or hydrogenated copra oil; and acyllactylates in which the acyl radical contains 8 to 20 carbon atoms. It is also possible to use alkyl-D-galactosiduronic acids and their salts, polyalkoxylated alkyl (C₆-C₂₄) ethercarboxylic acids, polyalkoxylated alkyl (C₆-C₂₄) arylethercarboxylic acids, polyalkoxylated alkyl(C₆-C₂₄)amidoethercarboxylic acids and their salts, particularly those containing from 2 to 50 alkylene oxide groups, especially ethylene oxide groups, and mixtures thereof.

Amphoteric or Zwitterionic Surfactants:

The amphoteric or zwitterionic surfactants, the nature of which is not critical within the framework of the present invention, can be the following in particular, without implying a limitation: aliphatic secondary or tertiary amine derivatives in which the aliphatic radical is a linear or branched chain containing 8 to 18 carbon atoms and at least one water-solubilizing anionic group (for example carboxylate, sulphonate, sulphate, phosphate or phosphonate); alkyl(C₈-C₂₀)betaines, sulphobetaines, alkyl (C₈-C₂₀) amidoalkyl (C₁-C₆) betaines or alkyl(C₈-C₂₀)amidoalkyl(C₁-C₆)sulphobetaines may also be mentioned.

Among the amine derivatives, there may be mentioned the products sold under the name MIRANOL, such as those described in patents U.S. Pat. Nos. 2,528,378 and US-2,781,354 and classified in the CTFA dictionary, 3rd edition, 1982, under the names Amphocarboxyglycinates and Amphocarboxypropionates. Their respective structures are:

R₂—CONHCH₂CH₂—N(R₃) (R₄) (CH₂COO⁻)

in which:

R₂ denotes the alkyl radical of an acid R₂—COOH present in hydrolysed copra oil, or a heptyl, nonyl or undecyl radical;

R₃ denotes a beta-hydroxyethyl group; and

R₄ denotes a carboxymethyl group; and

R₂′-CONHCH₂CH₂—N (B) (C)

in which:

B is —CH₂CH₂OX′;

C is —(CH₂) —Y′, where z=1 or 2;

X′ denotes the group —CH₂CH₂—COOH or a hydrogen atom;

Y′ denotes —COOH or the radical —CH₂—CHOH—SO₃H; and

R₂′ denotes the alkyl radical of an acid R₉—COOH present in copra oil or hydrolysed linseed oil, an alkyl radical, especially C₇, C₉, C₁₁ or C₁₃, a C₁₇ alkyl radical or its iso form, or an unsaturated C₁₇ radical.

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphodipropionate, Disodium Caprylamphodipropionate, Disodium Capryloamphodipropionate, Lauroamphodipropionic acid and Cocoamphodipropionic acid.

An example which may be mentioned is the cocoamphodiacetate marketed by RHODIA CHIMIE under the trade name MIRANOL® C2M concentrate.

Cationic Surfactants:

The following may be mentioned in particular, without implying a limitation, among the cationic surfactants: salts of optionally polyalkoxylated primary, secondary or tertiary fatty amines; quaternary ammonium salts such as tetraalkylammonium, alkylamidoalkyltrialkylammonium, trialkylbenzylammonium, trialkylhydroxyalkylammonium or alkylpyridinium chlorides or bromides; imidazoline derivatives; or cationic amine oxides.

The amounts of surfactants present in the composition according to the invention can vary from 0.01 to 40%, preferably from 0.5 to 30%, based on the total weight of the composition.

Preferably, according to the invention, the dyeing composition and/or the oxidizing composition can also contain more particularly at least one cationic polymer or at least one amphoteric polymer (other than those with a fatty chain according to the invention) in a proportion of at least 0.01% by weight, based on the total weight of the composition.

Cationic Polymers

In terms of the present invention, the expression “cationic polymer” denotes any polymer containing cationic groups and/or groups that can be ionized to cationic groups.

The cationic polymers which can be used according to the present invention can be selected from all those already known per se for improving the cosmetic properties of hair, especially those described in patent application EP-A-337 354 and French patents FR-2 270 846, 2 383 660, 2 598 611, 2 470 596 and 2 519 863.

The preferred cationic polymers are selected from those which comprise units containing primary, secondary, tertiary and/or quaternary amine groups that can either form part of the main polymer chain or be carried by a lateral substituent directly joined to said chain.

The cationic polymers used generally have a number-average molecular weight of between about 500 and 5.106, preferably of between about 103 and 3.106.

Polymers of the polyamine, polyaminoamide and quaternary polyammonium type may be mentioned more particularly among the cationic polymers. These are known products. They are described in particular in French patent no. 2 505 348 or 2 542 997. The following may be mentioned among said polymers:

(1) homopolymers or copolymers derived from acrylic or methacrylic acid esters or amides and containing at least one of the units of formulae (I), (II), (III) and (IV) below:

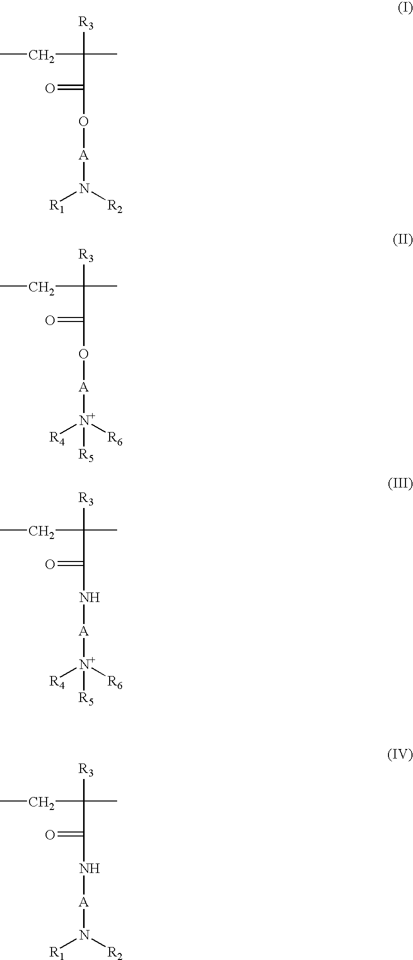

in which:

R₃, which are identical or different, denote a hydrogen atom or a CH₃ radical;

A, which are identical or different, are a linear or branched alkyl group having 1 to 6 carbon atoms, preferably 2 or 3 carbon atoms, or a hydroxyalkyl group having 1 to 4 carbon atoms;

R₄, R₅ and R₆, which are identical or different, are an alkyl group having from 1 to 18 carbon atoms or a benzyl radical, preferably an alkyl group having from 1 to 6 carbon atoms;

R₁ and R₂, which are identical or different, are hydrogen or an alkyl group having from 1 to 6 carbon atoms, preferably methyl or ethyl; and

X denotes an anion derived from a mineral or organic acid, such as a methosulphate anion or a halide such as chloride or bromide.

The polymers of family (1) can also contain one or more units derived from comonomers selectable from the family of acrylamides, methacrylamides, diacetonacrylamides, acrylamides and methacrylamides substituted on the nitrogen by lower alkyls (C₁-C₄), acrylic or methacrylic acids or esters, vinyllactams such as vinylpyrrolidone or vinylcaprolactam, and vinyl esters.

The following may thus be mentioned among these polymers of family (1):

copolymers of acrylamide and dimethylaminoethyl methacrylate quaternized with dimethyl sulphate or a dimethyl halide, such as the copolymer sold under the name HERCOFLOC by HERCULES;

the copolymers of acrylamide and methacryloyloxyethyltrimethylammonium chloride described e.g. in patent application EP-A-080 976 and sold under the name BINA QUAT P 100 by CIBA GEIGY;

the copolymer of acrylamide and methacryloyloxyethyltrimethylammonium methosulphate sold under the name RETEN by HERCULES;

quaternized or non-quaternized vinylpyrrolidone/dialkylaminoalkyl acrylate or methacrylate copolymers such as the products sold under the name “GAFQUAT” by ISP, for example “GAFQUAT 734” or “GAFQUAT 755”, or the products called “COPOLYMER 845, 958 and 937”; these polymers are described in detail in French patents 2.077.143 and 2.393.573;

dimethylaminoethyl methacrylate/vinylcaprolactam/vinylpyrrolidone terpolymers such as the product sold under the name GAFFIX VC 713 by ISP;

the vinylpyrrolidone/methacrylamidopropyldimethylamine copolymers marketed especially under the name STYLEZE CC 10 by ISP; and

quaternized vinylpyrrolidone/dimethylaminopropylmethacrylamide copolymers such as the product sold under the name “GAFQUAT HS 100” by ISP.

(2) the cellulose ether derivatives containing quaternary ammonium groups described in French patent 1 492 597, particularly the polymers marketed under the name “JR” (JR 400, JR 125, JR 30M) or “LR” (LR 400, LR 30M) by Union Carbide Corporation. These polymers are also defined in the CTFA dictionary as quaternary ammonium compounds of hydroxyethyl cellulose which has reacted with an epoxide substituted by a trimethylammonium group.

(3) cationic cellulose derivatives such as copolymers of cellulose or cellulose derivatives grafted with a water-soluble quaternary ammonium monomer, which are described especially in patent U.S. Pat. No. 4,131,576, such as hydroxyalkyl celluloses, like hydroxymethyl, hydroxyethyl or hydroxypropyl celluloses, grafted especially with a methacryloylethyltrimethylammonium, methacrylamidopropyltrimethylammonium or dimethyldiallylammonium salt.

The marketed products corresponding to this definition are more particularly the products sold under the names “Celquat L 200” and “Celquat H 100” by National Starch.

(4) the cationic polysaccharides described more particularly in patents U.S. Pat. No. 3,589,578 and 4 031 307, such as guar gums containing cationic trialkylammonium groups. Examples of cationic polysaccharides used are guar gums modified with a 2,3-epoxypropyltrimethylammonium salt (e.g. chloride). Such products are marketed especially under the trade names JAGUAR C13 S, JAGUAR C15, JAGUAR C17 and JAGUAR C162 by MEYHALL.

(5) polymers consisting of piperazinyl units and divalent alkylene or hydroxyalkylene radicals with linear or branched chains optionally interrupted by oxygen, sulphur or nitrogen atoms or by aromatic or heterocyclic rings, and the oxidation and/or quaternization products of these polymers. Such polymers are described especially in French patents 2.162.025 and 2.280.361.

(6) water-soluble polyaminoamides prepared in particular by the polycondensation of an acidic compound with a polyamine; these polyaminoamides can be crosslinked with an epihalogenohydrin, a diepoxide, a dianhydride, an unsaturated dianhydride, a bis-unsaturated derivative, a bis-halogenohydrin, a bis-azetidinium compound, a bis-halogenoacyldiamine or a bis-alkyl halide, or with an oligomer resulting from reaction with a difunctional compound that is reactive towards a bis-halogenohydrin, a bis-azetidinium compound, a bis-halogenoacyldiamine, a bis-alkyl halide, an epihalogenohydrin, a diepoxide or a bis-unsaturated derivative, the crosslinking agent being used in proportions ranging from 0.025 to 0.35 mol per amine group of the polyaminoamide; these polyaminoamides can be alkylated or, if they contain one or more tertiary amine groups, they can be quaternized. Such polymers are described especially in French patents 2.252.840 and 2.368.508.

(7) polyaminoamide derivatives resulting from the condensation of polyalkylenepolyamines with polycarboxylic acids, followed by alkylation with difunctional agents. Examples which may be mentioned are adipic acid/dialkylaminohydroxyalkyldialkylenetriamine polymers in which the alkyl radical contains from 1 to 4 carbon atoms and preferably denotes methyl, ethyl or propyl. Such polymers are described especially in French patent 1.583.363.

The adipic acid/dimethylaminohydroxypropyldiethylenetriamine polymers sold under the name “Cartaretine F, F4 or F8” by Sandoz may be mentioned more particularly among these derivatives.

(8) polymers obtained by the reaction of a polyalkylenepolyamine containing two primary amine groups and at least one secondary amine group with a dicarboxylic acid selected from diglycolic acid and saturated aliphatic dicarboxylic acids having from 3 to 8 carbon atoms, the molar ratio of polyalkylenepolyamine to dicarboxylic acid being between 0.8:1 and 1.4:1, and the resulting polyaminoamide being reacted with epichlorohydrin in a molar ratio of epichlorohydrin to secondary amine group of the polyaminoamide of between 0.5:1 and 1.8:1. Such polymers are described especially in American patents U.S. Pat. No. 3,227,615 and 2.961.347.

Polymers of this type are marketed in particular under the name “Hercosett 57” by Hercules Inc. or under the name “PD 170” or “Delsette 101” by Hercules in the case of the adipic acid/epoxypropyldiethylenetriamine copolymer.

(9) cyclic alkyldiallylamine or dialkyldiallylammonium polymers such as homopolymers or copolymers containing units of formula (V) or (VI) as the main constituent of the chain:

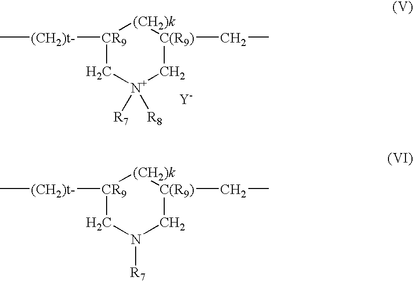

in which:

k and t are 0 or 1, the sum k+t being 1;

R₉ denotes a hydrogen atom or a methyl radical;

R₇ and R₈ independently of one another denote an alkyl group having from 1 to 22 carbon atoms, a hydroxyalkyl group in which the alkyl group preferably has 1 to 5 carbon atoms, or a lower amidoalkyl (C₁-C₄) group, or R₇ and R₈ can denote, together with the nitrogen atom to which they are attached, heterocyclic groups such as piperidinyl or morpholinyl; R₇ and R₈ independently of one another preferably denote an alkyl group having from 1 to 4 carbon atoms; and

Y⁻ is an anion such as bromide, chloride, acetate, borate, citrate, tartrate, bisulphate, bisulphite, sulphate or phosphate.

These polymers are described especially in French patent 2.080.759 and its certificate of addition 2.190.406.

The dimethyldiallylammonium chloride homopolymer sold under the name “Merquat 100” by Calgon (and its low weight-average molecular weight homologues) and the diallyldimethylammonium chloride/acrylamide copolymers marketed under the name “MERQUAT 550” may be mentioned more particularly among the polymers defined above.

(10) the quaternary diammonium polymer containing repeat units of the formula

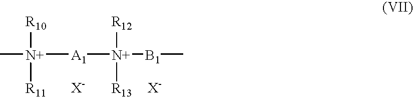

in which:

R₁₀, R₁₁, R₁₂ and R₁₃, which are identical or different, are aliphatic, alicyclic or arylaliphatic radicals containing from 1 to 20 carbon atoms or aliphatic lower hydroxyalkyl radicals, or R₁₀, R₁₁, R₁₂ and R₁₃ together or separately form, with the nitrogen atoms to which they are attached, heterocycles optionally containing a second heteroatom other than nitrogen, or R₁₀, R₁₁, R₁₂ and R₁₃ are a linear or branched C₁-C₆ alkyl radical substituted by a nitrile, ester, acyl or amide group or by a group —CO—O—R₁₄-D or —CO—NH—R₁₄-D, where R₁₄ is alkylene and D is a quaternary ammonium group;

A₁ and B₁ are polymethylene groups containing from 2 to 20 carbon atoms which can be linear or branched and saturated or unsaturated and can contain, bonded to or inserted in the main chain, one or more aromatic rings or one or more oxygen or sulphur atoms or sulphoxide, sulphone, disulphide, amino, alkylamino, hydroxyl, quaternary ammonium, ureido, amide or ester groups; and X⁻ denotes an anion derived from a mineral or organic acid;

A₁, R₁₀ and R₁₂ can form a piperazine ring with the two nitrogen atoms to which they are attached; moreover, if A₁ denotes a linear or branched, saturated or unsaturated alkylene or hydroxyalkylene radical, B₁ can also denote a group —(CH₂)ₙ—CO-D-OC—(CH₂)ₙ—, in which D denotes:

a) a glycol residue of the formula —O-Z-O—, where Z denotes a linear or branched hydrocarbon radical or a group of one of the following formulae:

—(CH₂—CH₂—O)ₓ—CH₂—CH₂—

—[CH₂—CH(CH₃)—O]_{y}—CH₂—CH(CH₃)—

where x and y denote an integer from 1 to 4, representing a defined and unique degree of polymerization, or any number from 1 to 4, representing a mean degree of polymerization;

b) a bis-secondary diamine residue such as a piperazine derivative;

c) a bis-primary diamine residue of the formula —NH—Y—NH—, where Y denotes a linear or branched hydrocarbon radical or the divalent radical

—CH₂—CH₂—S—S—CH₂—CH₂—; or

d) a ureylene group of the formula —NH—CO—NH—.

Preferably, X⁻ is an anion such as chloride or bromide. These polymers generally have a number-average molecular weight of between 1000 and 100,000.

Polymers of this type are described especially in French patents 2.320.330, 2.270.846, 2.316.271, 2.336.434 and 2.413.907 and patents U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

Polymers which can be used more particularly are those consisting of repeat units of formula (VIII) below:

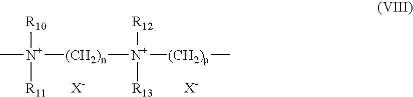

in which:

R₁₀, R₁₁, R₁₂ and R₁₃, which are identical or different, denote an alkyl or hydroxyalkyl radical having from about 1 to 4 carbon atoms;

n and p are integers varying from about 2 to 20; and X⁻ is an anion derived from a mineral or organic acid.

(11) quaternary polyammonium polymers consisting of repeat units of formula (IX):

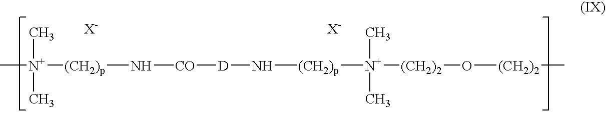

in which:

p is an integer varying from about 1 to 6;

D can be nothing or can represent a group —(CH2)ᵣ—CO—, in which r denotes a number equal to 4 or 7; and X⁻ is an anion.

Such polymers can be prepared by the processes described in U.S. Pat. Nos. 4,157,388, 4,702,906 and 4,719,282. They are described especially in patent application EP-A-122 324.

Examples which may be mentioned among these are the products “Mirapol A 15”, “Mirapol AD1”, “Mirapol AZ1” and “Mirapol 175” sold by Miranol.

(12) quaternary vinylpyrrolidone and vinylimidazole polymers such as the products marketed under the names Luviquat FC 905, FC 550 and FC 370 by BASF.

(13) polyamines such as Polyquart H sold by HENKEL, classified under the name “POLYETHYLENE GLYCOL (15) TALLOW POLYAMINE” in the CTFA dictionary.

(14) crosslinked polymers of methacryloyloxyalkyl (C₁-C₄)trialkyl (C₁-C₄) ammonium salts, such as polymers obtained by the homopolymerization of dimethylaminoethyl methacrylate quaternized with methyl chloride, or by the copolymerization of acrylamide and dimethylaminoethyl methacrylate quaternized with methyl chloride, the homopolymerization or copolymerization being followed by crosslinking with an olefinically unsaturated compound, particularly methylene-bis-acrylamide. More particularly, it is possible to use a crosslinked acrylamide/methacryloyloxyethyltrimethylammonium chloride copolymer (20/80 by weight) in the form of a dispersion containing 50% by weight of said copolymer in mineral oil. This dispersion is marketed under the name “SALCARE® SC 92” by ALLIED COLLOIDS. It is also possible to use a crosslinked methacryloyloxyethyltrimethylammonium chloride homopolymer containing about 50% by weight of the homopolymer in mineral oil or a liquid ester. These dispersions are marketed under the names “SALCARE® SC 95” and “SALCARE® SC 96” by ALLIED COLLOIDS.

Other cationic polymers which can be used within the framework of the invention are polyalkyleneimines, particularly polyethyleneimines, polymers containing vinylpyridine or vinylpyridinium units, condensates of polyamines and epichlorohydrin, quaternary polyureylenes and chitin derivatives.

Of all the cationic polymers capable of being used within the framework of the present invention, it is preferable to use the polymers of families (1), (9), (10), (11) and (14) and even more preferable to use polymers with the repeat units of formulae (W) and (U) below:

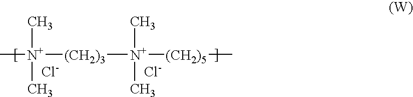

and especially those with a molecular weight of between 9500 and 9900, as determined by gel permeation chromatography;

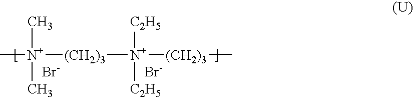

and especially those with a molecular weight of about 1200, as determined by gel permeation chromatography.

The concentration of cationic polymer in the composition according to the present invention can vary from 0.01 to 10% by weight, preferably from 0.05 to 5% and particularly preferably from 0.1 to 3%, based on the total weight of the composition.

Amphoteric Polymers Other Than Those of the Invention

The amphoteric polymers which can be used according to the present invention can be selected from polymers containing units K and M randomly distributed along the polymer chain, where K denotes a unit derived from a monomer containing at least one basic nitrogen atom and M denotes a unit derived from an acidic monomer containing one or more carboxylic or sulphonic acid groups, or alternatively K and M can denote groups derived from zwitterionic monomers of carboxybetaines or sulphobetaines;

K and M can also denote a cationic polymer chain containing primary, secondary, tertiary or quaternary amine groups, in which at least one of the amine groups carries a carboxylic or sulphonic acid group joined via a hydrocarbon radical, or alternatively K and M form part of a chain of a polymer with an α,β-dicarboxyethylene unit in which one of the carboxyl groups has been reacted with a polyamine containing one or more primary or secondary amine groups.

Of the amphoteric polymers corresponding to the definition given above, those which are more particularly preferred are selected from the following polymers:

(1) polymers resulting from the copolymerization of a monomer derived from a vinylic compound carrying a carboxyl group, such as, more particularly, acrylic acid, methacrylic acid, maleic acid or alpha-chloroacrylic acid, and a basic monomer derived from a substituted vinylic compound containing at least one basic atom, such as, more particularly, a dialkylaminoalkyl methacrylate or acrylate, a dialkylaminoalkylmethacrylamide or a dialkylaminoalkylacrylamide. Such compounds are described in American patent U.S. Pat. No. 3,836,537. The sodium acrylate/acrylamidopropyltrimethylammonium chloride copolymer sold under the name POLYQUART KE 3033 by HENKEL may also be mentioned.

The vinylic compound can also be a dialkyldiallylammonium salt such as dimethyldiallylammonium chloride. The copolymers of acrylic acid and the latter monomer are marketed under the names MERQUAT 280, MERQUAT 295 and MERQUAT PLUS 3330 by CALGON.

(2) polymers containing units derived from:

a) at least one monomer selected from acrylamides or methacrylamides substituted on the nitrogen by an alkyl radical,

b) at least one acidic comonomer containing one or more reactive carboxyl groups, and

c) at least one basic comonomer such as acrylic and methacrylic acid esters with primary, secondary, tertiary and quaternary amine substituents and the quaternization product of dimethylaminoethyl methacrylate with dimethyl or diethyl sulphate.

The N-substituted acrylamides or methacrylamides which are more particularly preferred according to the invention are those in which the alkyl radicals contain from 2 to 12 carbon atoms, and more particularly N-ethylacrylamide, N-tert-butylacrylamide, N-tert-octyl-acrylamide, N-octylacrylamide, N-decylacrylamide, N-dodecylacrylamide and the corresponding methacrylamides.

The acidic comonomers are selected more particularly from acrylic, methacrylic, crotonic, itaconic, maleic and fumaric acids and monoalkyl esters of maleic or fumaric acids or anhydrides in which the alkyl has 1 to 4 carbon atoms.

The preferred basic comonomers are aminoethyl, butylaminoethyl, N,N′-dimethylaminoethyl and N-tert-butylaminoethyl methacrylates.

The copolymers whose CTFA name (4th ed., 1991) is octylacrylamide/acrylate/butylaminoethyl methacrylate copolymers, such as the products sold under the name AMPHOMER or LOVOCRYL 47 by NATIONAL STARCH, are used in particular.

(3) crosslinked and alkylated polyaminoamides partially or totally derived from polyaminoamides of the general formula

[CO—R₁₉—CO-Z] (X)

in which:

R₁₉ is a divalent radical derived from a saturated dicarboxylic acid, from an aliphatic monocarboxylic or dicarboxylic acid with an ethylenic double bond, from an ester of these acids with a lower alkanol having 1 to 6 carbon atoms, or from a radical derived from the addition of any one of said acids onto a bis-primary or bis-secondary amine; and

Z denotes a radical of a bis-primary, mono-secondary or bis-secondary polyalkylenepolyamine and is preferably:

a) in proportions of 60 to 100 mol %, the radical

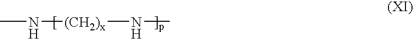

where x=2 and p=2 or 3, or x=3 and p=2, this radical being derived from diethylenetriamine, triethylenetetramine or dipropylenetriamine;

b) in proportions of 0 to 40 mol %, the above radical (XI) in which x=2 and p=1 and which is derived from ethylenediamine, or the following radical derived from piperazine:

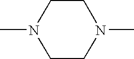

and

c) in proportions of 0 to 20 mol %, the radical —NH—(CH₂)₆—NH— derived from hexamethylenediamine, these polyaminoamines being crosslinked by the addition of a difunctional crosslinking agent selected from epihalogenohydrins, diepoxides, dianhydrides and bis-unsaturated derivatives, in an amount of 0.025 to 0.35 mol of crosslinking agent per amine group of the poly-aminoamide, and alkylated by reaction with acrylic acid, chloroacetic acid, an alkanesultone or their salts.

The saturated carboxylic acids are preferably selected from acids having 6 to 10 carbon atoms, such as adipic, 2,2,4-trimethyladipic, 2,4,4-trimethyladipic and terephthalic acids and acids with an ethylenic double bond, for example acrylic, methacrylic and itaconic acids. The alkanesultones used in the alkylation are preferably propanesultone or butanesultone, and the salts of the alkylating agents are preferably the sodium or potassium salts.

(4) polymers containing zwitterionic units of the formula

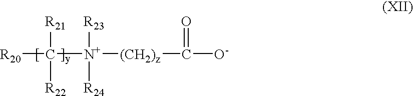

in which:

R₂₀ denotes a polymerizable unsaturated group such as an acrylate, methacrylate, acrylamide or methacrylamide group;

y and z are an integer from 1 to 3;

R₂₁ and R₂₂ are a hydrogen atom, methyl, ethyl or propyl; and

R₂₃ and R₂₄ are a hydrogen atom or an alkyl radical such that the sum of the carbon atoms in R₂₃ and R₂₄ does not exceed 10.

The polymers comprising such units can also contain units derived from non-zwitterionic monomers, such as dimethylaminoethyl or diethylaminoethyl acrylate or methacrylate, alkyl acrylates or methacrylates, acrylamides or methacrylamides, or vinyl acetate.

An example which may be mentioned is the butyl methacrylate/dimethylcarboxymethylammonioethyl methacrylate copolymer such as the product sold under the name DIAFORMER Z301 by SANDOZ.

(5) polymers derived from chitosan containing monomer units of formulae (XIII), (XIV) and (XV) below:

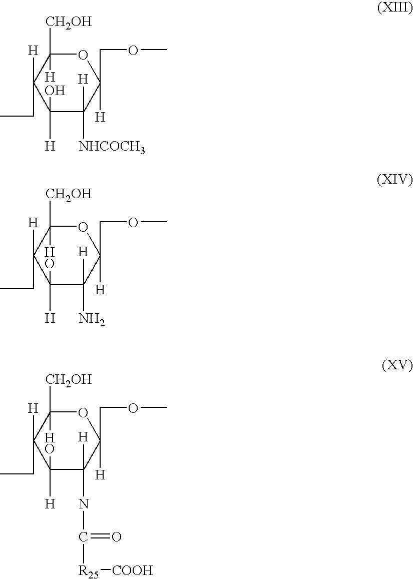

the unit (XIII) being present in proportions of between 0 and 30%, the unit (XIV) being present in proportions of between 5 and 50% and the unit F being present in proportions of between 30 and 90%, it being understood that, in said unit (XV), R₂₅ is a radical of the formula

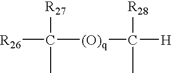

in which:

if q=0, R₂₆, R₂₇ and R₂₈, which are identical or different, are each a hydrogen atom, a methyl, hydroxyl, acetoxy or amino radical, a monoalkylamine radical or a dialkylamine radical optionally interrupted by one or more nitrogen atoms and/or optionally substituted by one or more amine, hydroxyl, carboxyl, alkylthio or sulphonic acid groups or an alkylthio radical in which the alkyl group carries an amino radical, at least one of the radicals R₂₆, R₂₇ and R₂₈ being a hydrogen atom in this case; or

if q=1, R₂₆, R₂₇ and R₂₈ are each a hydrogen atom, and the salts formed by these compounds with bases or acids.

(6) polymers derived from the N-carboxyalkylation of chitosan, such as Ncarboxymethylchitosan or the N-carboxybutylchitosan sold under the name “EVALSAN” by JAN DEKKER.

(7) polymers of general formula (XI), such as those described e.g. in French patent 1 400 366:

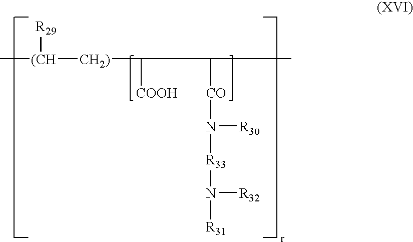

in which:

R₂₉ is a hydrogen atom or a CH₃O, CH₃CH₂O or phenyl radical;

R₃₀ denotes hydrogen or a lower alkyl radical such as methyl or ethyl;

R₃₁ denotes hydrogen or a lower alkyl radical such as methyl or ethyl; and

R₃₂ denotes a lower alkyl radical such as methyl or ethyl or a radical of the formula —R₃₃—N(R₃₁)₂, R₃₃ being a group —CH₂—CH₂—, —CH₂—CH₂—CH₂— or —CH₂—CH(CH₃)— and R₃₁ being as defined above,

and the higher homologues of these radicals containing up to 6 carbon atoms.

(8) amphoteric polymers of the type -D-X-D-X— selected from:

a) polymers obtained by reacting chloroacetic acid or sodium chloroacetate with compounds containing at least one unit of the formula

-D-X-D-X-D- (XVII)

where D denotes a radical

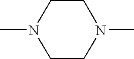

and X denotes the symbol E or E′, E or E′, which are identical or different, denoting a divalent radical which is an alkylene radical having a linear or branched chain containing up to 7 carbon atoms in the main chain, unsubstituted or substituted by hydroxyl groups and also capable of containing oxygen, nitrogen and sulphur atoms and 1 to 3 aromatic and/or heterocyclic rings, the oxygen, nitrogen and sulphur atoms being present in the form of ether, thioether, sulphoxide, sulphone, sulphonium, alkylamine, alkenylamine, hydroxyl, benzylamine, amine oxide, quaternary ammonium, amide, imide, alcohol, ester and/or urethane groups; and

b) polymers of the formula

-D-X-D-X— (XVIII)

where D denotes a radical

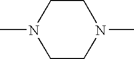

and X denotes the symbol E or E′ and denotes E′ at least once, E being defined as indicated above and E′ being a divalent radical which is an alkylene radical having a linear or branched chain containing up to 7 carbon atoms in the main chain, unsubstituted or substituted by one or more hydroxyl radicals and containing one or more nitrogen atoms, the nitrogen atom being substituted by an alkyl chain optionally interrupted by an oxygen atom and necessarily containing one or more carboxyl groups or one or more hydroxyl groups and converted to a betaine by reaction with chloroacetic acid or sodium chloroacetate.

(9) copolymers of alkyl(C₁-C₅) vinyl ether and maleic anhydride partially modified by semi-amidification with an N,N-dialkylaminoalkylamine such as N,N-dimethylaminopropylamine, or by semi-esterification with an N,N-dialkanolamine. These polymers can also contain other vinylic comonomers such as vinylcaprolactam.

The particularly preferred amphoteric polymers are those of family (1).

According to the invention, the amphoteric polymer(s) other than those with a fatty chain according to the invention can represent from 0.01% to 10% by weight, preferably from 0.05% to 5% by weight and particularly preferably from 0.1% to 3% by weight, based on the total weight of the composition.

The dyeing composition can also contain an effective amount of other agents previously known in oxidation dyeing, such as various customary adjuvants like sequestering agents, for example EDTA and etidronic acid, UV filters, waxes, volatile or non-volatile cyclic, linear or branched silicones that may or may not be organomodified (especially with amine groups), preservatives, ceramides, pseudoceramides, vegetable, mineral or synthetic oils, vitamins or provitamins like panthenol, opacifiers, etc.

Said composition can also contain reducing agents or antioxidants. These can be selected in particular from sodium sulphite, thioglycolic acid, thiolactic acid, sodium bisulphite, dehydroascorbic acid, hydroquinone, 2-methylhydroquinone, tert-butylhydroquinone and homogentisic acid, generally being present in amounts ranging from about 0.05 to 3% by weight, based on the total weight of the composition.

Of course, those skilled in the art will take care to ensure that any complementary compound(s) mentioned above will be chosen in such a way that the advantageous properties intrinsically attached to the dyeing composition according to the invention are totally or substantially unaffected by the envisaged addition(s).

In the ready-to-use composition or in the oxidizing composition, the oxidizing agent is preferably selected from urea peroxide, alkali metal bromates or ferricyanides, and per salts such as perborates and persulphates. The use of hydrogen peroxide is particularly preferred. This oxidizing agent advantageously consists of a hydrogen peroxide solution whose strength can vary more particularly from about 1 to 40 volumes and even more preferably from about 5 to 40.

Other oxidizing agents which can be used are one or more redox enzymes such as laccases, peroxidases and oxidoreductases with 2 electrons (such as uricase), if necessary in the presence of their respective donor or cofactor.

The pH of the ready-to-use composition applied to keratin fibres [composition resulting from the mixing of the dyeing composition and the oxidizing composition] is generally between the values 4 and 11. It is preferably between 6 and 10 and can be adjusted to the desired value with acidifying or alkalizing agents well known in the state of the art of keratin fibre dyeing.

Examples of alkalizing agents which may be mentioned are aqueous ammonia, alkali metal carbonates, alkanolamines such as mono-, di- and triethanolamines and their derivatives, sodium and potassium hydroxides and the compounds of formula (IV) below:

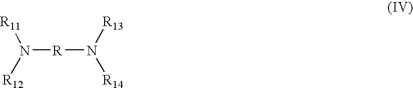

in which:

R is a propylene radical optionally substituted by a hydroxyl group or a C₁-C₄ alkyl radical; and

R₁₁, R₁₂, R₁₃ and R₁₄, which are identical or different, are a hydrogen atom or a C₁-C₄ alkyl or C₁-C₄ hydroxyalkyl radical.

Examples of conventional acidifying agents are mineral or organic acids such as hydrochloric acid, orthophosphoric acid, carboxylic acids like tartaric acid, citric acid or lactic acid, or sulphonic acids.

The dyeing process according to the invention preferably consists in applying a mixture, prepared at the time of use (ready-to-use composition) from the dyeing and oxidizing compositions described above, to the dry or wet keratin fibres, leaving it to act for a period of time that varies preferably from about 1 to 60 minutes and particularly preferably from about 5 to 45 minutes, rinsing the fibres, optionally shampooing them and then rinsing them again, and drying them.

In said process the dyeing and/or oxidizing compositions can also contain at least one cationic polymer or amphoteric polymer (other than those with a fatty chain) and at least one, preferably non-ionic surfactant.

A concrete example illustrating the invention will now be given without thereby implying a limitation.

### EXAMPLE

The following dyeing composition according to the invention was prepared:

| Dyeing composition | % by weight |
|---|---|
| Non-ionic surfactants (ethoxylated | 32.5 |
| fatty alcohols) | |
| Glycerol | 3 |
| Oleic acid | 2 |
| Monoethanolamine | 0.47 |
| Oleyl alcohol | 1.8 |
| Fatty amide | 4 |
| Amphoteric polymer with a fatty chain | 0.3 AS* |
| according to the invention [MAPTAC | |
| (49)/AA(49)/SMA(1.9994)/MBA | |
| (0.0006)] | |
| Cationic polymer | 2.4 |
| Amphoteric polymer other than those of | 1.2 |
| the invention | |
| Vegetable oils | 0.6 |
| Sequestering agent, antioxidant, | qs |
| reducing agent, perfume | |
| Aqueous ammonia (20% NH₃) | 8 |
| 2-Methyl-5-hydroxyethylaminophenol | 0.86 |
| p-Aminophenol | 0.41 |
| 4-Amino-2-hydroxytoluene | 0.57 |
| 6-Hydroxyindole | 0.068 |
| p-Phenylenediamine | 0.49 |
| Resorcinol | 0.1 |
| Water | qsp 100 |

*As denotes Active Substance
MAPTAC = Methylacrylamidopropyltrimethylammonium chloride
AA = Acrylic acid
SMA = Stearyl methacrylate
MBA = N,N′-methylene-bis-acrylamide

After it has been mixed with hydrogen peroxide and applied to the hair, this dyeing composition affords a strong, chromatic and durable shade of low selectivity.

1. Composition for the oxidation dyeing of keratin fibres, particularly human keratin fibres such as hair, comprising, in an appropriate dyeing medium, at least one oxidation dyestuff precursor and/or coupler selected from paraphenylenediamines, double bases, paraminophenols, metaphenylenediamines, metaminophenols, metadiphenols, bases and heterocyclic couplers, and the addition salts of these compounds with an acid, and at least one amphoteric polymer with at least one fatty chain having from 8 to 30 carbon atoms.

2. Composition according to claim 1, characterized in that the amphoteric polymer with at least one fatty chain also contains at least one non-cyclic cationic unit.

3. Composition according to claim 1 or 2, characterized in that said amphoteric polymer comprises 1 to 20 mol % of monomer with a fatty chain, based on the total number of moles of monomers.

4. Composition according to any one of claims 1 to 3, characterized in that the amphoteric polymers comprise:
1) at least one monomer of formula (Ia) or (Ib) below: 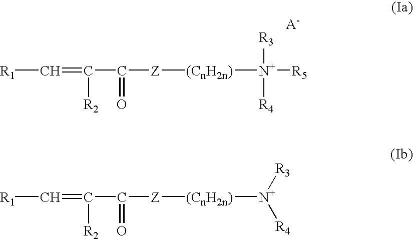
in which:
R₁ and R₂, which are identical or different, are a hydrogen atom or a methyl radical;
R₃, R₄ and R₅, which are identical or different, are a linear or branched alkyl radical having from 1 to 30 carbon atoms;
Z is an NH group or an oxygen atom;
n is an integer from 2 to 5; and
A⁻ is an anion derived from an organic or mineral acid;
2) at least one monomer of formula (II)
R₆—CH═CR₇—COOH (II)
in which:
R₆ and R₇, which are identical or different, are a hydrogen atom or a methyl radical; and
3) at least one monomer of formula (III):
R₆—CH═CR₇—COXR₈ (III)
in which:
R₆ and R₇, which are identical or different, are a hydrogen atom or a methyl radical;
X denotes an oxygen or nitrogen atom; and
R₈ denotes a linear or branched alkyl radical having from 1 to 30 carbon atoms,
at least one of the monomers of formula (Ia), (Ib) or (III) containing at least one fatty chain having from 8 to 30 carbon atoms.

5. Composition according to claim 4, characterized in that the monomers of formulae (Ia) and (Ib) are selected from the group comprising dimethylaminoethyl methacrylate, dimethylaminoethyl acrylate, diethylaminoethyl methacrylate, diethylaminoethyl acrylate, dimethylaminopropyl methacrylate, dimethylaminopropyl acrylate, dimethylaminopropylmethacrylamide and dimethylaminopropylacrylamide, which are optionally quaternized.

6. Composition according to either of claims 4 and 5, characterized in that the monomer of formula (Ia) is acrylamidopropyltrimethylammonium chloride or methacrylamidopropyltrimethylammonium chloride.

7. Composition according to claim 4, characterized in that the monomer of formula (II) is selected from the group comprising acrylic acid, methacrylic acid, crotonic acid and 2-methylcrotonic acid.

8. Composition according to claim 4, characterized in that the monomer of formula (III) is selected from the group comprising C₁₂-C₂₂ and preferably C₁₆-C₁₈ alkyl acrylates or methacrylates.

9. Composition according to any one of claims 1 to 8, characterized in that said amphoteric polymers with a fatty chain are selected from acrylic acid/(meth)acrylamidopropyltrimethylammonium chloride/stearyl methacrylate terpolymers.

10. Composition according to claim 1, characterized in that the amphoteric polymers with a fatty chain are selected from polymers derived from polyaspartic acid.

11. Composition according to any one of the preceding claims, characterized in that the amphoteric polymer with a fatty chain is used in an amount of between 0.05 and 10% by weight, based on the total weight of the composition.

12. Composition according to claim 11, characterized in that the amphoteric polymer with a fatty chain is used in an amount of between 0.1 and 5% by weight, based on the total weight of the composition.

13. Composition according to claim 1, characterized in that the paraphenylenediamines are selected from those of formula (I) below and their acid addition salts: 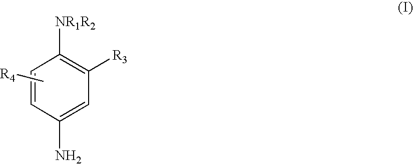
in which:
R₁ is a hydrogen atom, a C₁-C₄ alkyl radical, a C₁-C₄ monohydroxyalkyl radical, a C₂-C₄ polyhydroxyalkyl radical, an alkoxy(C₁-C₄)alkyl(C₁-C₄) radical or a C₁-C₄ alkyl radical substituted by a nitrogen, phenyl or 4′-aminophenyl group;
R₂ is a hydrogen atom, a C₁-C₄ alkyl radical, a C₁-C₄ monohydroxyalkyl radical, a C₂-C₄ polyhydroxyalkyl radical, an alkoxy(C₁-C₄)alkyl(C₁-C₄) radical or a C₁-C₄ alkyl radical substituted by a nitrogen group;
R₃ is a hydrogen atom, a halogen atom such as a chlorine atom, or a C₁-C₄ alkyl, sulpho, carboxyl, C₁-C₄ monohydroxyalkyl, C₁-C₄ hydroxyalkoxy, C₁-C₄ acetylaminoalkoxy, C₁-C₄ mesylaminoalkoxy or C₁-C₄ carbamoylaminoalkoxy radical; and
R₄ is a hydrogen or halogen atom or a C₁-C₄ alkyl radical,
it also being possible for R₁ and R₂ to form, with the nitrogen atom which carries them, a 5- or 6-membered nitrogen heterocycle optionally substituted by one or more alkyl, hydroxyl or ureido groups.

14. Composition according to claim 13, characterized in that the nitrogen radicals are selected from amino, monoalkyl(C₁-C₄)amino, dialkyl(C₁-C₄) amino, trialkyl (C₁-C₄)amino, monohydroxyalkyl(C₁-C₄)amino, imidazolinium and ammonium radicals.

15. Composition according to either of claims 13 and 14, characterized in that the paraphenylenediamines are selected from paraphenylenediamine, paratoluylenediamine, 2-chloroparaphenylenediamine, 2,3-dimethylparaphenylenediamine, 2,6-dimethylparaphenylenediamine, 2,6-diethylparaphenylenediamine, 2,5-dimethylparaphenylenediamine, N,N-dimethylparaphenylenediamine, N,N-diethylparaphenylenediamine, N,N-dipropylparaphenylenediamine, 4-amino-N,N-diethyl-3-methylaniline, N,N-bis(β-hydroxyethyl)paraphenylenediamine, 4-N,N-bis-(β-hydroxyethyl)amino-2-methylaniline, 4-N,N-bis(β-hydroxyethyl)amino-2-chloroaniline, 2-β-hydroxyethylparaphenylenediamine, 2-fluoroparaphenylenediamine, 2-isopropylparaphenylenediamine, N-(β-hydroxypropyl)paraphenylenediamine, 2-hydroxymethylparaphenylenediamine, N,N-dimethyl-3-methylparaphenylenediamine, N,N-(ethyl, β-hydroxyethyl)paraphenylenediamine, N-(β,γ-dihydroxypropyl)paraphenylenediamine, N-(4′-aminophenyl)paraphenylenediamine, N-phenylparaphenylenediamine, 2-β-hydroxyethyloxyparaphenylenediamine, 2-β-acetylaminoethyloxyparaphenylenediamine, N-(β-methoxyethyl)paraphenylenediamine, 2-methyl-1-N-β-hydroxyethylparaphenylenediamine and their acid addition salts.

16. Composition according to claim 1, characterized in that the double bases are selected from those of formula (II) below and their acid addition salts: 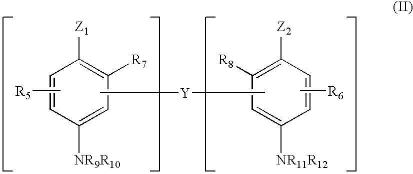
in which:
Z₁ and Z₂, which are identical or different, are a hydroxyl or —NH₂ radical which can be substituted by a C₁-C₄ alkyl radical or by a linking arm Y;
the linking arm Y is a linear or branched alkylene chain containing from 1 to 14 carbon atoms which can be interrupted or terminated by one or more nitrogen groups and/or by one or more heteroatoms such as oxygen, sulphur or nitrogen atoms, and optionally substituted by one or more hydroxyl or C₁-C₆ alkoxy radicals;
R₅ and R₆ are a hydrogen or halogen atom, a C₁-C₄ alkyl, C₁-C₄ monohydroxyalkyl, C₂-C₄ polyhydroxyalkyl or C₁-C₄ aminoalkyl radical or a linking arm Y; and
R₇, R₈, R₉, R₁₀, R₁₁ and R₁₂, which are identical or different, are a hydrogen atom, a linking arm Y or a C₁-C₄ alkyl radical,
it being understood that the compounds of formula (II) contain only one linking arm Y per molecule.

17. Composition according to claim 16, characterized in/that, in formula (II), the nitrogen groups are selected from amino, monoalkyl(C₁-C₄)amino, dialkyl (C₁-C₄) amino, trialkyl (C₁-C₄)amino, monohydroxyalkyl (C₁-C₄) amino, imidazolinium and ammonium radicals.

18. Composition according to either of claims 16 and 17, characterized in that the double bases are selected from N,N′-bis(β-hydroxyethyl)-N,N′-bis (4′-aminophenyl)-1,3-diaminopropanol, N,N′-bis (β-hydroxyethyl) —N,N′-bis (4′-aminophenyl) ethylenediamine,
N,N′-bis (4-aminophenyl)tetramethylenediamine, N,N′-bis (β-hydroxyethyl) —N,N′-bis (4-aminophenyl)tetramethylenediamine, N,N′-bis (4-methylaminophenyl)-tetramethylenediamine, N,N′-bis (ethyl)-N,N′-bis (4′-amino, 3′-methylphenyl)ethylenediamine, 1,8-bis (2,5-diaminophenoxy)-3,5-dioxaoctane and their acid addition salts.

19. Composition according to claim 1, characterized in that the paraminophenols are selected from those of formula (III) below and their acid addition salts: 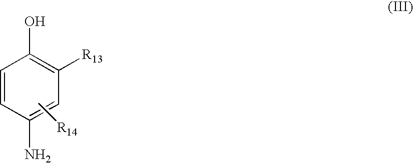
in which:
R₁₃ is a hydrogen atom, a halogen atom such as fluorine, or a C₁-C₄ alkyl, C₁-C₄ monohydroxyalkyl, alkoxy(C₁-C₄)— alkyl (C₁-C₄), C₁-C₄ aminoalkyl or hydroxyalkyl (C₁-C₄)— aminoalkyl(C₁-C₄) radical; and
R₁₄ is a hydrogen atom, a halogen atom such as fluorine, or a C₁-C₄ alkyl, C₁-C₄ monohydroxyalkyl, C₂-C₄ polyhydroxyalkyl, C₁-C₄ aminoalkyl, C₁-C₄ cyanoalkyl or alkoxy (C₁-C₄) alkyl (C₁-C₄) radical.

20. Composition according to claim 19, characterized in that the paraminophenols are selected from 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and their acid addition salts.

21. Composition according to claim 1, characterized in that the heterocyclic bases are selected from pyridine derivatives, pyrimidine and pyrazolopyrimidine derivatives, pyrazole derivatives and their acid addition salts.

22. Composition according to claim 21, characterized in that the pyridine derivatives are selected from 2,5-diaminopyridine, 2-(4-methoxyphenyl) amino-3-aminopyridine, 2, 3-diamino-6-methoxypyridine, 2- (β-methoxyethyl) amino-3-amino-6-methoxypyridine, 3,4-diaminopyridine and their acid addition salts.

23. Composition according to claim 21, characterized in that the pyrimidine derivatives are selected from 2,4,5,6-tetraminopyrimidine, 4-hydroxy-2,5,6-triaminopyrimidine, 2-hydroxy-4,5,6-triaminopyrimidine, 2,4-dihydroxy-5,6-diaminopyrimidine and 2,5,6-triaminopyrimidine, and the pyrazolopyrimidine derivatives are selected from pyrazolo[1,5-a]pyrimidine-3,7-diamine, 2,5-dimethylpyrazolo[1,5-a]pyrimidine-3,7-diamine, pyrazolo-[1,5-a]pyrimidine-3,5-diamine, 2,7-dimethylpyrazolo[1,5-a]pyrimidine-3,5-diamine, 3-aminopyrazolo-[1,5-a]pyrimidin-7-ol, 3-aminopyrazolo[1,5-a]pyrimidin-5-ol, 2-(3-aminopyrazolo[1,5-a]pyrimidin-7-ylamino)-ethanol, 2-(7-aminopyrazolo[1,5-a]pyrimidin-3-ylamino)-ethanol, 2-[(3-aminopyrazolo[1,5-a]pyrimidin-7-yl)(2-hydroxyethyl)amino]ethanol, 2-[(7-aminopyrazolo[1,5-a]-pyrimidin-3-yl)(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine, 2,5, N7,N7-tetramethylpyrazolo[1,5-a]pyrimidine-3,7-diamine, 3-amino-5-methyl-7-imidazolylpropylaminopyrazolo[1,5-a]pyrimidine and their addition salts, and their tautomeric forms if there is a tautomeric equilibrium, and their acid addition salts.

24. Composition according to claim 21, characterized in that the pyrazole derivatives are selected from 4,5-diamino-1-methylpyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole,
4,5-diamino-1-(β-hydroxyethyl)-3-methylpyrazole, 4,5-diamino-1-(β-hydroxyethyl)pyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxy methylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5- (2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole,
3,5-diamino-4-(β-hydroxyethyl)amino-1-methylpyrazole and their acid addition salts.

25. Composition according to any one of the preceding claims, characterized in that the oxidation dyestuff precursors are present in concentrations ranging from 0.0005 to 12% by weight, based on the total weight of the composition.

26. Composition according to claim 1, characterized in that the couplers are selected from metaphenylenediamines, metaminophenols, metadiphenols, heterocyclic couplers and their acid addition salts.

27. Composition according to claim 26, characterized in that the couplers are selected from 2,4-diamino-1-(β-hydroxyethyloxy)benzene, 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2-amino-4-(β-hydroxyethylamino)-1-methoxybenzene, 1,3-diaminobenzene, 1,3-bis(2,4-diaminophenoxy)propane, sesamol, 1-amino-2-methoxy-4,5-methylenedioxybenzene, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one, 1-phenyl-3-methylpyrazol-5-one, 2-amino-3-hydroxypyridine, 3,6-dimethylpyrazolo[3,2-c]-1,2,4-triazole, 2,6-dimethylpyrazolo[1,5-b]-1,2,4-triazole and their acid addition salts.

28. Composition according to any one of the preceding claims, characterized in that the couplers are present in concentrations ranging from 0.0001 to 10% by weight, based on the total weight of the composition.

29. Composition according to any one of claims 1 and 13 to 28, characterized in that the acid addition salts of the oxidation dyestuff precursors and of the couplers are selected from hydrochlorides, hydrobromides, sulphates, tartrates, lactates and acetates.

30. Composition according to any one of the preceding claims, characterized in that it also contains direct dyestuffs in a proportion of 0.001 to 20% by weight, based on the total weight of the composition.

31. Composition according to any one of the preceding claims, characterized in that it also contains at least one cationic polymer or at least one amphoteric polymer other than the amphoteric polymers with a fatty chain as defined in any one of claims 1 to 10.

32. Composition according to claim 31, characterized in that the cationic polymer is a quaternary polyammonium compound consisting of repeat units of formula (W) below:

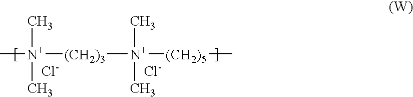

33. Composition according to claim 31, characterized in that the cationic polymer is a quaternary polyammonium compound consisting of repeat units of formula (U) below:

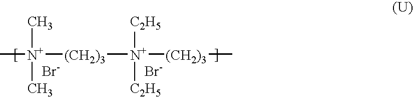

34. Composition according to claim 31, characterized in that the amphoteric polymer is a copolymer comprising at least acrylic acid and a dimethyldiallylammonium chloride as monomers.

35. Composition according to any one of claims 31 to 34, characterized in that the cationic polymer(s) or the amphoteric polymers other than those with a fatty chain according to the invention represent from 0.01% to 10% by weight, based on the total weight of the composition.

36. Composition according-to any one of the preceding claims, characterized in that it contains at least one surfactant selected from anionic, cationic, non-ionic or amphoteric surfactants.

37. Composition according to claim 36, characterized in that the surfactant(s) is(are) non-ionic.

38. Composition according to claim 36 or 37, characterized in that the surfactants represent 0.01 to 40% by weight, based on the total weight of the composition.

39. Composition according to any one of the preceding claims, characterized in that it also contains at least one reducing agent in amounts ranging from 0.05 to 3% by weight, based on the total weight of the composition.

40. Ready-to-use composition according to any one of the preceding claims, characterized in that it also contains an oxidizing agent.

41. Composition according to claim 40, characterized in that it has a pH ranging from 4 to 11.

42. Composition according to claim 40, characterized in that the oxidizing agent is selected from hydrogen peroxide, urea peroxide, alkali metal bromates or ferricyanides, persalts and redox enzymes, optionally with their respective donor or cofactor.

43. Composition according to claim 42, characterized in that the oxidizing agent is hydrogen peroxide.

44. Composition according to claim 43, characterized in that the oxidizing agent is a hydrogen peroxide solution whose strength varies from 1 to 40 volumes.

45. Process for dyeing keratin fibres, particularly human keratin fibres such as hair, characterized in that it consists in applying to the fibres at least one dyeing composition containing, in an appropriate dyeing medium, at least one oxidation dyestuff precursor and/or coupler selected from paraphenylenediamines, double bases, paraminophenols, metaphenylenediamines, metaminophenols, metadiphenols, bases and heterocyclic couplers, and the addition salts of these compounds with an acid,
the colour being developed at alkaline, neutral or acidic pH with the aid of an oxidizing composition containing at least one oxidizing agent, which is mixed with the dyeing composition at the time of use or is applied sequentially without intermediate rinsing, at least one amphoteric polymer with at least one fatty chain, as defined according to claims 1 to 10, being present in the dyeing composition, in the oxidizing composition or in each of said compositions.

46. Process according to claim 30, characterized in that it consists in applying, to the dry or wet keratin fibres, the ready-to-use composition prepared at the time of use from the dyeing and oxidizing compositions, leaving it to act for a period of time varying from about 1 to 60 minutes, preferably from 5 to 45 minutes, rinsing the fibres, optionally shampooing them and then rinsing them again, and drying them.

47. Device with several compartments, or kit, for dyeing keratin fibres, particularly human keratin fibres such as hair, characterized in that it comprises at least two compartments, one of which comprises a dyeing composition containing, in an appropriate dyeing medium, at least one oxidation dyestuff precursor and/or coupler selected from paraphenylenediamines, double bases, paraminophenols, metaphenylenediamines, metaminophenols, metadiphenols, bases and heterocyclic couplers, and the addition salts of these compounds with an acid,
and another of which comprises an oxidizing composition containing, in an appropriate dyeing medium, an oxidizing agent, an amphoteric polymer with at least one fatty chain, as defined in any one of claims 1 to 10, being present in the dyeing composition, in the oxidizing composition or in each of the two compositions.
